SEQUENCE LISTING (28 sequences)

SEQ ID NO: 1 - protein, 777 aa, Homo sapiens
  misc_feature: Origin human brain-derived clone 0M007
MNANKDERLKARSQDFHLFPALMMLSMTMLFLPVTGTLKQNIPRLKLTYKDLLLSNSCIPFLGSSEGLDFQTLLLDEERGRLLLGAKDHIFLLSLVDLNKNFKKIYWPAAKERVELCKLAGKDANTECANFIRVLQPYNKTHIYVCGTGAFHPICGYIDLGVYKEDIIFKLDTRNLESGRLKCPFDPQQPFASVMTDEYLYSGTASDFLGKDTAFTRSLGPTHDHHYIRTDISEHYWLNGAKFIGTFFIPDTYNPDDDKIYFFFRESSQEGSTSDKTILSRVGRVCKNDVGGQRSLINKWTTFLKARLICSIPGSDGADTYFDELQDIYLLPTRDERNPVVYGVFTTTSSIFKGSAVCVYSMADIRAVFNGPYAHKESADHRWVQYDGRIPYPRPGTCPSKTYDPLIKSTRDFPDDVISFIKRHSVMYKSVYPVAGGPTFKRINVDYRLTQIVVDHVIAEDGQYDVMFLGTDIGTVLKVVSISKEKWNMEEVVLEELQIFKHSSIILNMELSLKQQQLYIGSRDGLVQLSLHRCDTYGKACADCCLARDPYCAWDGNACSRYAPTSKRRARRQDVKYGDPITQCWDIEDSISHETADEKVIFGIEFNSTFLECIPKSQQATIKWYIQRSGDEHREELKPDERIIKTEYGLLIRSLQKKDSGMYYCKAQEHTFIHTIVKLTLNVIENEQMENTQRAEHEEGQVKDLLAESRLRYKDYIQILSSPNFSLDQYCEQMWHREKRRQRNKGGPKWKHMQEMKKKRNRRHHRDLDELPRAVAT

SEQ ID NO: 2 - DNA, 2331 bp, Homo sapiens
atgaatgctaataaagatgaaagacttaaagccagaagccaagattttcacctttttcctgctttgatgatgctaagcatgaccatgttgtttcttccagtcactggcactttgaagcaaaatattccaagactcaagctaacctacaaagacttgctgctttcaaatagctgtattccctttttgggttcatcagaaggactggattttcaaactcttctcttagatgaggaaagaggcaggctgctcttgggagccaaagaccacatctttctactcagtctggttgacttaaacaaaaattttaagaagatttattggcctgctgcaaaggaacgggtggaattatgtaaattagctgggaaagatgccaatacagaatgtgcaaatttcatcagagtacttcagccctataacaaaactcacatatatgtgtgtggaactggagcatttcatccaatatgtgggtatattgatcttggagtctacaaggaggatattatattcaaactagacacacgtaatttggagtctggcagactgaaatgtcctttcgatcctcagcagccttttgcttcagtaatgacagatgagtacctctactctggaacagcttctgatttccttggcaaagatactgcattcactcgatcccttgggcctactcatgaccaccactacatcagaactgacatttcagagcactactggctcaatggagcaaaatttattggaactttcttcataccagacacctacaatccagatgatgataaaatatatttcttctttcgtgaatcatctcaagaaggcagtacctccgataaaaccatcctttctcgagttggaagagtttgtaagaatgatgtaggaggacaacgcagcctgataaacaagtggacgacttttcttaaggccagactgatttgctcaattcctggaagtgatggggcagatacttactttgatgagcttcaagatatttatttactccccacaagagatgaaagaaatcctgtagtatatggagtctttactacaaccagctccatcttcaaaggctctgctgtttgtgtgtatagcatggctgacatcagagcagtttttaatggtccatatgctcataaggaaagtgcagaccatcgttgggtgcagtatgatgggagaattccttatccacggcctggtacatgtccaagcaaaacctatgacccactgattaagtccacccgagattttccagatgatgtcatcagtttcataaagcggcactctgtgatgtataagtccgtatacccagttgcaggaggaccaacgttcaagagaatcaatgtggattacagactgacacagatagtggtggatcatgtcattgcagaagatggccagtacgatgtaatgtttcttggaacagacattggaactgtcctcaaagttgtcagcatttcaaaggaaaagtggaatatggaagaggtagtgctggaggagttgcagatattcaagcactcatcaatcatcttgaacatggaattgtctctgaagcagcaacaattgtacattggttcccgagatggattagttcagctctccttgcacagatgcgacacttatgggaaagcttgcgcagactgttgtcttgccagagacccctactgtgcctgggatggaaatgcatgctctcgatatgctcctacttctaaaaggagagctagacgccaagatgtaaaatatggcgacccaatcacccagtgctgggacatcgaagacagcattagtcatgaaactgctgatgaaaaggtgatttttggcattgaatttaactcaacctttctggaatgtatacctaaatcccaacaagcaactattaaatggtatatccagaggtcaggggatgagcatcgagaggagttgaagcccgatgaaagaatcatcaaaacggaatatgggctactgattcgaagtttgcagaagaaggattctgggatgtattactgcaaagcccaggagcacactttcatccacaccatagtgaagctgactttgaatgtcattgagaatgaacagatggaaaatacccagagggcagagcatgaggaggggcaggtcaaggatctattggctgagtcacggttgagatacaaagactacatccaaatccttagcagcccaaacttcagcctcgaccagtactgcgaacagatgtggcacagggagaagcggagacagagaaacaaggggggcccaaagtggaagcacatgcaggaaatgaagaagaaacgaaatcgaagacatcacagagacctggatgagctccctagagctgtagccacg

SEQ ID NO: 3 - DNA, 3880 bp, Homo sapiens
  misc_feature: Origin human brain-derived clone 0M007
caccttaccaactgcagatcttgggactcatcagcctcaataattatattaaattaacaccatttgaaagagaacattgttttcatcatgaatgctaataaagatgaaagacttaaagccagaagccaagattttcacctttttcctgctttgatgatgctaagcatgaccatgttgtttcttccagtcactggcactttgaagcaaaatattccaagactcaagctaacctacaaagacttgctgctttcaaatagctgtattccctttttgggttcatcagaaggactggattttcaaactcttctcttagatgaggaaagaggcaggctgctcttgggagccaaagaccacatctttctactcagtctggttgacttaaacaaaaattttaagaagatttattggcctgctgcaaaggaacgggtggaattatgtaaattagctgggaaagatgccaatacagaatgtgcaaatttcatcagagtacttcagccctataacaaaactcacatatatgtgtgtggaactggagcatttcatccaatatgtgggtatattgatcttggagtctacaaggaggatattatattcaaactagacacacgtaatttggagtctggcagactgaaatgtcctttcgatcctcagcagccttttgcttcagtaatgacagatgagtacctctactctggaacagcttctgatttccttggcaaagatactgcattcactcgatcccttgggcctactcatgaccaccactacatcagaactgacatttcagagcactactggctcaatggagcaaaatttattggaactttcttcataccagacacctacaatccagatgatgataaaatatatttcttctttcgtgaatcatctcaagaaggcagtacctccgataaaaccatcctttctcgagttggaagagtttgtaagaatgatgtaggaggacaacgcagcctgataaacaagtggacgacttttcttaaggccagactgatttgctcaattcctggaagtgatggggcagatacttactttgatgagcttcaagatatttatttactccccacaagagatgaaagaaatcctgtagtatatggagtctttactacaaccagctccatcttcaaaggctctgctgtttgtgtgtatagcatggctgacatcagagcagtttttaatggtccatatgctcataaggaaagtgcagaccatcgttgggtgcagtatgatgggagaattccttatccacggcctggtacatgtccaagcaaaacctatgacccactgattaagtccacccgagattttccagatgatgtcatcagtttcataaagcggcactctgtgatgtataagtccgtatacccagttgcaggaggaccaacgttcaagagaatcaatgtggattacagactgacacagatagtggtggatcatgtcattgcagaagatggccagtacgatgtaatgtttcttggaacagacattggaactgtcctcaaagttgtcagcatttcaaaggaaaagtggaatatggaagaggtagtgctggaggagttgcagatattcaagcactcatcaatcatcttgaacatggaattgtctctgaagcagcaacaattgtacattggttcccgagatggattagttcagctctccttgcacagatgcgacacttatgggaaagcttgcgcagactgttgtcttgccagagacccctactgtgcctgggatggaaatgcatgctctcgatatgctcctacttctaaaaggagagctagacgccaagatgtaaaatatggcgacccaatcacccagtgctgggacatcgaagacagcattagtcatgaaactgctgatgaaaaggtgatttttggcattgaatttaactcaacctttctggaatgtatacctaaatcccaacaagcaactattaaatggtatatccagaggtcaggggatgagcatcgagaggagttgaagcccgatgaaagaatcatcaaaacggaatatgggctactgattcgaagtttgcagaagaaggattctgggatgtattactgcaaagcccaggagcacactttcatccacaccatagtgaagctgactttgaatgtcattgagaatgaacagatggaaaatacccagagggcagagcatgaggaggggcaggtcaaggatctattggctgagtcacggttgagatacaaagactacatccaaatccttagcagcccaaacttcagcctcgaccagtactgcgaacagatgtggcacagggagaagcggagacagagaaacaaggggggcccaaagtggaagcacatgcaggaaatgaagaagaaacgaaatcgaagacatcacagagacctggatgagctccctagagctgtagccacgtagttttctacttaatttaaagaaaagaattccttacctataaaaacattgccttctgttttgtatatcccttatagtaattcataaatgcttcccatggagttttgctaaggcacaagacaataatctgaataagacaatatgtgatgaatataagaaagggcaaaaaattcatttgaaccagttttccaagaacaaatcttgcacaagcaaagtataagaattatcctaaaaatagggggtttacagttgtaaatgttttatgttttgagttttggaatttattgtcatgtaaatagttgagctaagcaagccccgaatttgatagtgtataaggtgctttattccctcgaatgtccattaagcatggaatttaccatgcagttgtgctatgttcttatgaacagatatatcattcctattgagaaccagctaccttgtggtagggaataagaggtcagacacaaattaagacaactcccattatcaacaggaactttctcagtgagccattcactcctggagaatggtataggaatttggagaggtgcattatttctttctggccactggggttaaatttagtgtactacaacattgatttactgaagggcactaatgtttcccccaggatttctattgactagtcaggagtaacaggttcacagagagaagttggtgcttagttatgtgttttttagagtatatactaagctctacagggacagaatgcttaataaatactttaataagatatgggaaaatattttaataaaacaaggaaaacataatgatgtataatgcatcctgatgggaaggcatgcagatgggatttgttagaagacagaaggaaagacagccataaattctggctttggggaaaactcatatccccatgaaaaggaagaacaatcacaaataaagtgagagtaatgtaatggagctcttttcactagggtataagtagctgccaatttgtaattcatctgttaaaaaaaatctagattataacaaactgctagcaaaatctgaggaaacataaattcttctgaagaatcataggaagagtagacattttatttataaccaatgatatttcagtatatattttctctcttttaaaaaatatttatcatactctgtatattatttctttttactgcctttattctctcctgtatattggattttgtgattatatttgagtgaataggagaaaacaatatataacacacagagaattaagaaaatgacatttctggggagtggggatatatatttgttgaataacagaacgagtgtaaaattttaacaacggaaagggttaaattaactctttgacatcttcactcaaccttttctcattgctgagttaatctgttgtaattgtagtattgtttttgtaatttaacaataaataagcctgctacatgtaaaaagaaccaaaaaaaaaaaaaaaaaa

SEQ ID NO: 4 - protein, 356 aa, Homo sapiens
  misc_feature: Origin human brain-derived clone 0MB096
MHHQWLLLAACFWVIFMFMVASKFITLTFKDPDVYSAKQEFLFLTTMPEVRKLPEEKHIPEELKPTGKELPDSQLVQPLVYMERLELIRNVCRDDALKNLSHTPVSKFVLDRIFVCDKHKILFCQTPKVGNTQWKKVLIVLNGAFSSIEEIPENVVHDHEKNGLPRLSSFSDAEIQKRLKTYFKFFIVRDPFERLISAFKDKFVHNPRFEPWYRHEIAPGIIRKYRRNRTETRGIQFEDFVRYLGDPNHRWLDLQFGDHIIHWVTYVELCAPCEIMYSVIGHHETLEDDAPYILKEAGIDHLVSYPTIPPGITVYNRTKVEHYFLGISKRDIRRLYARFEGDFKLFGYQKPDFLLN

SEQ ID NO: 5 - DNA, 1068 bp, Homo sapiens
atgcaccaccagtggcttctgctggccgcatgcttttgggtgattttcatgttcatggtggctagcaagttcatcacgttgacctttaaagacccagatgtgtacagtgccaaacaggagtttctgttcctgacaaccatgccggaagtgaggaagttgccagaagagaagcacattcctgaggaactgaagccaactgggaaggagcttccagacagccagctcgttcagcccctggtctacatggagcgcctggaactcatcagaaacgtctgcagggatgatgccctgaagaatctctcgcacactcctgtctccaagtttgtcctggaccgaatatttgtctgtgacaagcacaagattcttttctgccagactcccaaagtgggcaacacccagtggaagaaagtgctgattgttctaaatggagcattttcttccattgaggagatccccgaaaacgtggtgcacgaccacgagaagaacggccttcctcggctctcttccttcagtgatgcagaaattcagaagcgattgaaaacatacttcaagttttttattgtaagagatcccttcgaaagacttatttctgcatttaaggataaatttgttcacaatccccggtttgagccttggtacaggcatgagattgctcctggcatcatcagaaaatacaggaggaaccggacagagacccgggggatccagtttgaagatttcgtgcgctacctcggcgatccgaaccacagatggctagaccttcagtttggggaccacatcattcactgggtgacgtatgtagagctctgtgctccctgtgagataatgtacagtgtgattggacaccacgagaccctggaggacgatgccccatacatcttaaaagaggctggcattgaccacctggtgtcatacccgactatccctccgggcattaccgtgtataacagaaccaaggtggagcactatttcctgggcatcagcaaacgagacatccgacgcctgtatgcccgtttcgaaggggactttaagctctttgggtaccagaaaccagactttttgctaaac

SEQ ID NO: 6 - DNA, 2479 bp, Homo sapiens
  misc_feature: Origin human brain-derived clone 0MB096
acaacatgcaccaccagtggcttctgctggccgcatgcttttgggtgattttcatgttcatggtggctagcaagttcatcacgttgacctttaaagacccagatgtgtacagtgccaaacaggagtttctgttcctgacaaccatgccggaagtgaggaagttgccagaagagaagcacattcctgaggaactgaagccaactgggaaggagcttccagacagccagctcgttcagcccctggtctacatggagcgcctggaactcatcagaaacgtctgcagggatgatgccctgaagaatctctcgcacactcctgtctccaagtttgtcctggaccgaatatttgtctgtgacaagcacaagattcttttctgccagactcccaaagtgggcaacacccagtggaagaaagtgctgattgttctaaatggagcattttcttccattgaggagatccccgaaaacgtggtgcacgaccacgagaagaacggccttcctcggctctcttccttcagtgatgcagaaattcagaagcgattgaaaacatacttcaagttttttattgtaagagatcccttcgaaagacttatttctgcatttaaggataaatttgttcacaatccccggtttgagccttggtacaggcatgagattgctcctggcatcatcagaaaatacaggaggaaccggacagagacccgggggatccagtttgaagatttcgtgcgctacctcggcgatccgaaccacagatggctagaccttcagtttggggaccacatcattcactgggtgacgtatgtagagctctgtgctccctgtgagataatgtacagtgtgattggacaccacgagaccctggaggacgatgccccatacatcttaaaagaggctggcattgaccacctggtgtcatacccgactatccctccgggcattaccgtgtataacagaaccaaggtggagcactatttcctgggcatcagcaaacgagacatccgacgcctgtatgcccgtttcgaaggggactttaagctctttgggtaccagaaaccagactttttgctaaactaatgcataagacctatgaattcaaatatctttattagacctggggctaaccaggtgaagatctgagcccagaaatgacccttcctccaccacacccctcctttgaggacgcccggggtctcccacaggcctgtgagttgcctcggcatatgacgcagaaccccaactgttacaacttagtttggatgtaagatgctctgaggaccctgcccacacccctgcgtgcattaggatgtcgctggcctttgctcacctcagaggggagaaaaggctaaagatttgcagtttgacagcccagcagggaggaagcatcacacagcgttaggagccgtttccttcaggtgttaaggaaggggatgcccctgaggttctcctggctagtcagggtggcttcacccatcactggtgggttgcaggaacagcacccaggactctgaggagggacagagaagcaagggggctgctgaaatcgcagagacttttgcagcatcagatctgaggagtaaaacggcacctctggccttcatcttggtgctgcgacaattgtggaggcaaagcattctttctgtgactattttgttcctgtagacagtcagcgatggccagagggtggtgtggtgtccaggggtccatctttccagaatccatgcctgtgtaatgctggtccatgcttctgaacctgtgtctgccaagcgcctatttcattcagcacaagacatacgattttagaaggtgaggggaggggaggctttttctacctgagaaggggagtgtctttgagggccttaaaaggaccatggcccaggaatgggggcgctggttgggcttggagctcaggctgctgtggatcccggcgcatcagttctgacttgccttacctgggtggacagcagtgaatctccacctgtcttctccagggagctcccatgttggggctgaagacgagcaggggcaacctgccagcatcacagaattcagtgtagtttatacatttcgattcctttcatctcagcaaaatgggcactgccagagccatttctgatcacaccaccatcctggaccatgtgactggaaggtgggtaaccaagttcaccagcaataaaacccagcgcccaggtagcctccagcagtgcggcttcctggcaacaaggtaggccctggtgcagggcaagccgcagcgaccatttcagataccgtccacagccaggaccgctgagaactgggacagtttcctgggatgagtgccagcctgagcctgcatggtgccgccgagcccggggtggaggagggagccaggcttcgcttcaaggcggcctctaccttttctcagaatggtttcctgattgtgtcaatgtgaaagttaaataaaatttatgtgccaaacctgaaaaaaaaaaaaaaaaaaa

SEQ ID NO: 7 - protein, 343 aa, Homo sapiens
  misc_feature: Origin human bone marrow stroma cell HAS 303-derived clone 0AF038-Leu
MWSHLNRLLFWSIFSSVTCRKAVLDCEAMKTNEFPSPCLDSKTKVVMKGQNVSMFCSHKNKSLQITYSLFRRKTHLGTQDGKGEPAIFNLSITEAHESGPYKCKAQVTSCSKYSRDFSFTIVDPVTSPVLNIMVIQTETDRHITLHCLSVNGSLPINYTFFENHVAISPAISKYDREPAEFNLTKKNPGEEEEYRCEAKNRLPNYATYSHPVTMPSTGGDSCPFCLKLLLPGLLLLLVVIILILAFWVLPKYKTRKAMRNNVPRDRGDTAMEVGIYANILEKQAKEESVPEVGSRPCVSTAQDEAKHSQELQYATPVFQEVAPREQEACDSYKSGYVYSELNF

SEQ ID NO: 8 - DNA, 1029 bp, Homo sapiens
atgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttc

SEQ ID NO: 9 - DNA, 1370 bp, Homo sapiens
  misc_feature: Origin human bone marrow stroma cell HAS 303-derived clone 0AF038-Leu
gggagaatgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcagggtaaggatgctttttatgaagctgatttccatgaacaaaaagcaaacttgaggctgaggcgggtggatcacagggtcaggagatcaagaccatcctggctaacacgatgaaaccccgtctctactaaaaaatacaaaaattagccaggtgtggtggtgtgtgtgtgtagtcccagctactcgggaggctgaggcaggagaatcgcttgagcccgggaggcagaggttgcagtgagccaagatcgtgccactgcactacagcctgggcgacaagagcaagacttcatctcaaaaaaaaaaaaaaa

SEQ ID NO: 10 - protein, 343 aa, Homo sapiens
  misc_feature: Origin human bone marrow stroma cell HAS 303-derived clone 0AF038-Pro
MWSHLNRLLFWSIFSSVTCRKAVLDCEAMKTNEFPSPCLDSKTKVVMKGQNVSMFCSHKNKSLQITYSLFRRKTHPGTQDGKGEPAIFNLSITEAHESGPYKCKAQVTSCSKYSRDFSFTIVDPVTSPVLNIMVIQTETDRHITLHCLSVNGSLPINYTFFENHVAISPAISKYDREPAEFNLTKKNPGEEEEYRCEAKNRLPNYATYSHPVTMPSTGGDSCPFCLKLLLPGLLLLLVVIILILAFWVLPKYKTRKAMRNNVPRDRGDTAMEVGIYANILEKQAKEESVPEVGSRPCVSTAQDEAKHSQELQYATPVFQEVAPREQEACDSYKSGYVYSELNF

SEQ ID NO: 11 - DNA, 1029 bp, Homo sapiens
atgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacccgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttc

SEQ ID NO: 12 - DNA, 1370 bp, Homo sapiens
  misc_feature: Origin human bone marrow stroma cell HAS 303-derived clone 0AF038-Pro
gggagaatgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacccgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcagggtaaggatgctttttatgaagctgatttccatgaacaaaaagcaaacttgaggctgaggcgggtggatcacagggtcaggagatcaagaccatcctggctaacacgatgaaaccccgtctctactaaaaaatacaaaaattagccaggtgtggtggtgtgtgtgtgtagtcccagctactcgggaggctgaggcaggagaatcgcttgagcccgggaggcagaggttgcagtgagccaagatcgtgccactgcactacagcctgggcgacaagagcaagacttcatctcaaaaaaaaaaaaaaa

SEQ ID NO: 13 - protein, 118 aa, Homo sapiens
  misc_feature: Origin human embryonal liver-derived clone 0R087H
MRLFLWNAVLTLFVTSLIGALIPEPEVKIEVLQKPFICHRKTKGGDLMLVHYEGYLEKDGSLFHSTHKHNNGQPIWFTLGILEALKGWDQGLKGMCVGEKRKLIIPPALGYGKEGKVF

SEQ ID NO: 14 - DNA, 354 bp, Homo sapiens
atgaggcttttcttgtggaacgcggtcttgactctgttcgtcacttctttgattggggctttgatccctgaaccagaagtgaaaattgaagttctccagaagccattcatctgccatcgcaagaccaaaggaggggatttgatgttggtccactatgaaggctacttagaaaaggacggctccttatttcactccactcacaaacataacaatggtcagcccatttggtttaccctgggcatcctggaggctctcaaaggttgggaccagggcttgaaaggaatgtgtgtaggagagaagagaaagctcatcattcctcctgctctgggctatggaaaagaaggaaaagtcttt

SEQ ID NO: 15 - DNA, 1875 bp, Homo sapiens
  misc_feature: Origin human embryonal liver-derived clone 0R087H
cctgaacttgtctgaagcccttgtccgtaagccttgaactacgttcttaaatctatgaagtcgagggacctttcgctgcttttgtagggacttctttccttgcttcagcaacatgaggcttttcttgtggaacgcggtcttgactctgttcgtcacttctttgattggggctttgatccctgaaccagaagtgaaaattgaagttctccagaagccattcatctgccatcgcaagaccaaaggaggggatttgatgttggtccactatgaaggctacttagaaaaggacggctccttatttcactccactcacaaacataacaatggtcagcccatttggtttaccctgggcatcctggaggctctcaaaggttgggaccagggcttgaaaggaatgtgtgtaggagagaagagaaagctcatcattcctcctgctctgggctatggaaaagaaggaaaagtcttttagtacatgcttgcatgcctcttttggaaagataccagttttatcaacaacctagcgcatgtcacatctctgtctagatctgaaatggtaaaattcccccagaaagtacactgatatttaatattgatctcctggagattcgaaatggaccaagatcccatgaatcattccaagaaatggatcttaatgatgactggaaactctctaaagatgaggttaaagcatatttaaagaaggagtttgaaaaacatggtgcggtggtgaatgaaagtcatcatgatgctttggtggaggatatttttgataaagaagatgaagacaaagatgggtttatatctgccagagaatttacatataaacacgatgagttatagagatacatctacccttttaatatagcactcatctttcaagagagggcagtcatctttaaagaacattttatttttatacaatgttctttcttgctttgttttttatttttatatattttttctgactcctatttaaagaaccccttaggtttctaagtacccatttctttctgataagttattgggaagaaaaagctaattggtctttgaatagaagacttctggacaatttttcactttcacagatatgaagctttgttttactttctcacttataaatttaaaatgttgcaactgggaatataccacgacatgagaccaggttatagcacaaattagcaccctatatttctgcttccctctattttctccaagttagaggtcaacatttgaaaagccttttgcaatagcccaaggcttgctattttcatgttataatgaaatagtttatgtgtaactggctctgagtctctgcttgaggaccagaggaaaatggttgttggacctgacttgttaatggctactgctttactaaggagatgtgcaatgctgaagttagaaacaaggttaatagccaggcatggtggctcatgcctgtaatcccagcactttgggaggctgaggcgggcggatcacctgaggttgggagttcgagaccagcctgaccaacacggagaaaccctatctctactaaaaatacaaaagtagccgggcgtggtgatgcgtgcctgtaatcccagctacccaggaaggctgaggcggcagaatcacttgaacccggaggcggaggttgcggtaagccgagatcacctccagcctggacactctgtctcgaaaaaaagaaaagaaacacggttaataacatataaatatgtatgcattgagacatgctacctaggacttaagctgatgaagcttggctcctagtgattggtggcctattatgataaataggacaaatcatttatgtgtgagtttctttgtaataaaatgtatcaatatgttaaaaaaaaaaaaaaaaaaaaaaaaaaaaaa

SEQ ID NO: 16 - protein, 377 aa, Homo sapiens
  misc_feature: Origin human glioblastoma cell line T98G-derived clone 0A004FG
MDSALSDPHNGSAEAGGPTNSTTRPPSTPEGIALAYGSLLLMALLPIFFGALRSVRCARGKNASDMPETITSRDAARFPIIASCTLLGLYLFFKIFSQEYINLLLSMYFFVLGILALSHTISPFMNKFFPASFPNRQYQLLFTQGSGENKEEIINYEFDTKDLVCLGLSSIVGVWYLLRKHWIANNLFGLAFSLNGVELLHLNNVSTGCILLGGLFIYDVFWVFGTNVMVTVAKSFEAPIKLVFPQDLLEKGLEANNFAMLGLGDVVIPGIFIALLLRFDISLKKNTHTYFYTSFAAYIFGLGLTIFIMHIFKHAQPALLYLVPACIGFPVLVALAKGEVTEMFSYEESNPKDPAAVTESKEGTEASASKGLEKKEK

SEQ ID NO: 17 - DNA, 1131 bp, Homo sapiens
atggactcggccctcagcgatccgcataacggcagtgccgaggcaggcggccccaccaacagcactacgcggccgccttccacgcccgagggcatcgcgctggcctacggcagcctcctgctcatggcgctgctgcccatcttcttcggcgccctgcgctccgtacgctgcgcccgcggcaagaatgcttcagacatgcctgaaacaatcaccagccgggatgccgcccgcttccccatcatcgccagctgcacactcttggggctctacctctttttcaaaatattctcccaggagtacatcaacctcctgctgtccatgtatttcttcgtgctgggaatcctggccctgtcccacaccatcagccccttcatgaataagttttttccagccagctttccaaatcgacagtaccagctgctcttcacacagggttctggggaaaacaaggaagagatcatcaattatgaatttgacaccaaggacctggtgtgcctgggcctgagcagcatcgttggcgtctggtacctgctgaggaagcactggattgccaacaacctttttggcctggccttctcccttaatggagtagagctcctgcacctcaacaatgtcagcactggctgcatcctgctgggcggactcttcatctacgatgtcttctgggtatttggcaccaatgtgatggtgacagtggccaagtccttcgaggcaccaataaaattggtgtttccccaggatctgctggagaaaggcctcgaagcaaacaactttgccatgctgggacttggagatgtcgtcattccagggatcttcattgccttgctgctgcgctttgacatcagcttgaagaagaatacccacacctacttctacaccagctttgcagcctacatcttcggcctgggccttaccatcttcatcatgcacatcttcaagcatgctcagcctgccctcctatacctggtccccgcctgcatcggttttcctgtcctggtggcgctggccaagggagaagtgacagagatgttcagttatgaggagtcaaatcctaaggatccagcggcagtgacagaatccaaagagggaacagaggcatcagcatcgaaggggctggagaagaaagagaaa

SEQ ID NO: 18 - DNA, 1612 bp, Homo sapiens
  misc_feature: Origin human glioblastoma cell line T98G-derived clone 0A004FG
cacgtcacttcctgttgccttaggggaacgtggctttccctgcagagccggtgtctccgcctgcgtccctgctgcagcaaccggagctggagtcggatcccgaacgcaccctcgccatggactcggccctcagcgatccgcataacggcagtgccgaggcaggcggccccaccaacagcactacgcggccgccttccacgcccgagggcatcgcgctggcctacggcagcctcctgctcatggcgctgctgcccatcttcttcggcgccctgcgctccgtacgctgcgcccgcggcaagaatgcttcagacatgcctgaaacaatcaccagccgggatgccgcccgcttccccatcatcgccagctgcacactcttggggctctacctctttttcaaaatattctcccaggagtacatcaacctcctgctgtccatgtatttcttcgtgctgggaatcctggccctgtcccacaccatcagccccttcatgaataagttttttccagccagctttccaaatcgacagtaccagctgctcttcacacagggttctggggaaaacaaggaagagatcatcaattatgaatttgacaccaaggacctggtgtgcctgggcctgagcagcatcgttggcgtctggtacctgctgaggaagcactggattgccaacaacctttttggcctggccttctcccttaatggagtagagctcctgcacctcaacaatgtcagcactggctgcatcctgctgggcggactcttcatctacgatgtcttctgggtatttggcaccaatgtgatggtgacagtggccaagtccttcgaggcaccaataaaattggtgtttccccaggatctgctggagaaaggcctcgaagcaaacaactttgccatgctgggacttggagatgtcgtcattccagggatcttcattgccttgctgctgcgctttgacatcagcttgaagaagaatacccacacctacttctacaccagctttgcagcctacatcttcggcctgggccttaccatcttcatcatgcacatcttcaagcatgctcagcctgccctcctatacctggtccccgcctgcatcggttttcctgtcctggtggcgctggccaagggagaagtgacagagatgttcagttatgaggagtcaaatcctaaggatccagcggcagtgacagaatccaaagagggaacagaggcatcagcatcgaaggggctggagaagaaagagaaatgatgcggctggtgcccgagcctctcagggccagaccagacagatgggggctgggcccacacaggcgtgcaccggtagagggcacaggaggccaagggcagctccaggacagggcagggggcagcaggatacctccagccaggcctctgtggcctctgtttccttctccctttcttggccctcctctgctcctccccacaccctgcaggcaaaagaaacccccagcttcccccctccccgggagccaggtgggaaaagtgggtgtgatttttagattttgtattgtggactgattttgcctcacattaaaaactcatcccatggccagggcgggccactgtgctcctgaaaaaaaaaaaaaaaaa

SEQ ID NO: 19 - protein, 377 aa, Homo sapiens
  misc_feature: Origin human glioblastoma cell line T98G-derived clone 0A004LD
MDSALSDPHNGSAEAGGPTNSTTRPPSTPEGIALAYGSLLLMALLPIFFGALRSVRCARGKNASDMPETITSRDAARFPIIASCTLLGLYLFFKIFSQEYINLLLSMYFFVLGILALSHTISPFMNKFFPASLPNRQYQLLFTQGSGENKEEIINYEFDTKDLVCLGLSSIVDVWYLLRKHWIANNLFGLAFSLNGVELLHLNNVSTGCILLGGLFIYDVFWVFGTNVMVTVAKSFEAPIKLVFPQDLLEKGLEANNFAMLGLGDVVIPGIFIALLLRFDISLKKNTHTYFYTSFAAYIFGLGLTIFIMHIFKHAQPALLYLVPACIGFPVLVALAKGEVTEMFSYEESNPKDPAAVTESKEGTEASASKGLEKKEK

SEQ ID NO: 20 - DNA, 1131 bp, Homo sapiens
atggactcggccctcagcgatccgcataacggcagtgccgaggcaggcggccccaccaacagcactacgcggccgccttccacgcccgagggcatcgcgctggcctacggcagcctcctgctcatggcgctgctgcccatcttcttcggcgccctgcgctccgtacgctgcgcccgcggcaagaatgcttcagacatgcctgaaacaatcaccagccgggatgccgcccgcttccccatcatcgccagctgcacactcttggggctctacctctttttcaaaatattctcccaggagtacatcaacctcctgctgtccatgtatttcttcgtgctgggaatcctggccctgtcccacaccatcagccccttcatgaataagttttttccagccagccttccaaatcgacagtaccagctgctcttcacacagggttctggggaaaacaaggaagagatcatcaattatgaatttgacaccaaggacctggtgtgcctgggcctgagcagcatcgttgacgtctggtacctgctgaggaagcactggattgccaacaacctttttggcctggccttctcccttaatggagtagagctcctgcacctcaacaatgtcagcactggctgcatcctgctgggcggactcttcatctacgatgtcttctgggtatttggcaccaatgtgatggtgacagtggccaagtccttcgaggcaccaataaaattggtgtttccccaggatctgctggagaaaggcctcgaagcaaacaactttgccatgctgggacttggagatgtcgtcattccagggatcttcattgccttgctgctgcgctttgacatcagcttgaagaagaatacccacacctacttctacaccagctttgcagcctacatcttcggcctgggccttaccatcttcatcatgcacatcttcaagcatgctcagcctgccctcctatacctggtccccgcctgcatcggttttcctgtcctggtggcgctggccaagggagaagtgacagagatgttcagttatgaggagtcaaatcctaaggatccagcggcagtgacagaatccaaagagggaacagaggcatcagcatcgaaggggctggagaagaaagagaaa

SEQ ID NO: 21 - DNA, 1612 bp, Homo sapiens
  misc_feature: Origin human glioblastoma cell line T98G-derived clone 0A004LD
cacgtcacttcctgttgccttaggggaacgtggctttccctgcagagccggtgtctccgcctgcgtccctgctgcagcaaccggagctggagtcggatcccgaacgcaccctcgccatggactcggccctcagcgatccgcataacggcagtgccgaggcaggcggccccaccaacagcactacgcggccgccttccacgcccgagggcatcgcgctggcctacggcagcctcctgctcatggcgctgctgcccatcttcttcggcgccctgcgctccgtacgctgcgcccgcggcaagaatgcttcagacatgcctgaaacaatcaccagccgggatgccgcccgcttccccatcatcgccagctgcacactcttggggctctacctctttttcaaaatattctcccaggagtacatcaacctcctgctgtccatgtatttcttcgtgctgggaatcctggccctgtcccacaccatcagccccttcatgaataagttttttccagccagccttccaaatcgacagtaccagctgctcttcacacagggttctggggaaaacaaggaagagatcatcaattatgaatttgacaccaaggacctggtgtgcctgggcctgagcagcatcgttgacgtctggtacctgctgaggaagcactggattgccaacaacctttttggcctggccttctcccttaatggagtagagctcctgcacctcaacaatgtcagcactggctgcatcctgctgggcggactcttcatctacgatgtcttctgggtatttggcaccaatgtgatggtgacagtggccaagtccttcgaggcaccaataaaattggtgtttccccaggatctgctggagaaaggcctcgaagcaaacaactttgccatgctgggacttggagatgtcgtcattccagggatcttcattgccttgctgctgcgctttgacatcagcttgaagaagaatacccacacctacttctacaccagctttgcagcctacatcttcggcctgggccttaccatcttcatcatgcacatcttcaagcatgctcagcctgccctcctatacctggtccccgcctgcatcggttttcctgtcctggtggcgctggccaagggagaagtgacagagatgttcagttatgaggagtcaaatcctaaggatccagcggcagtgacagaatccaaagagggaacagaggcatcagcatcgaaggggctggagaagaaagagaaatgatgcggctggtgcccgagcctctcagggccagaccagacagatgggggctgggcccacacaggcgtgcaccggtagagggcacaggaggccaagggcagctccaggacagggcagggggcagcaggatacctccagccaggcctctgtggcctctgtttccttctccctttcttggccctcctctgctcctccccacaccctgcaggcaaaagaaacccccagcttcccccctccccgggagccaggtgggaaaagtgggtgtgatttttagattttgtattgtggactgattttgcctcacattaaaaactcatcccatggccagggcgggccactgtgctcctgaaaaaaaaaaaaaaaaa

SEQ ID NO: 22 - DNA, 35 bp, Artificial Sequence
  Primer
cgattgaattctagacctgcctcgagnnnnnnnnn

SEQ ID NO: 23 - DNA, 27 bp, Artificial Sequence
  Primer 0M007-F3
aactgcagatcttgggactcatcagcc

SEQ ID NO: 24 - DNA, 28 bp, Artificial Sequence
  Primer 0M007-F2
aagaggacattgttttcatcatggatgc

SEQ ID NO: 25 - DNA, 27 bp, Artificial Sequence
  Primer 0MB096-F1
acaacatgcaccaccagtggcttctgc

SEQ ID NO: 26 - DNA, 28 bp, Artificial Sequence
  Primer 0AF038-F1
agaatgtggagccatttgaacaggctcc

SEQ ID NO: 27 - DNA, 27 bp, Artificial Sequence
  Primer 0R087H-F1
tgaagcccttgtccgtaagccttgaac

SEQ ID NO: 28 - DNA, 24 bp, Artificial Sequence
  Primer 0A004-F1
atgcacatcttcaagcatgctcag

(19) United States
(12) Patent Application Publication
(10) Pub. No.: US 2010/0285527 A1
(43) Pub. Date: Nov. 11, 2010

(54) Novel Polypeptide, cDNA Encoding the Same, and Use Thereof

(75) Inventors: Daikichi FUKUSHIMA, Osaka (JP); Shiro Shibayama, Osaka (JP); Hideaki Tada, Osaka (JP)

Correspondence Address: SUGHRUE-265550, 2100 PENNSYLVANIA AVE. NW, WASHINGTON, DC, 20037-3213, US

(73) Assignee: ONO PHARMACEUTICAL CO., LTD., Osaka (JP)

(21) Appl. No.: 12/772,617

(22) Filed: May 3, 2010

Related U.S. Application Data
(63) Continuation of application No. 11/593,037, filed on Nov. 6, 2006, now abandoned.
Application No. 11/593,037 is a continuation of application No. 10/404,438, filed on Apr. 2, 2003, now abandoned.
Application No. 10/404,438 is a continuation of application No. 09/582,419, filed on Jun. 26, 2000, now abandoned.

(30) Foreign Application Priority Data
Dec. 26, 1997 (JP) P. HEI. 9/358811

(51) Int. Cl. C12P 21/02; C07K 14/00; C07H 21/04; C12N 15/63; C12N 1/19

(52) U.S. Cl. 435/69.1; 530/350; 536/23.1; 435/320.1; 435/254.21

(57) ABSTRACT

Novel polypeptides produced by a human adult brain tissue, a cell line derived therefrom, a cell line derived from human bone marrow and a human umbilical cord venous endothelial cell line; a process for producing these polypeptides; cDNAs encoding the polypeptides; fragments hybridizable selectively with the cDNA sequences; replication or expression plasmids having the cDNAs integrated thereinto; host cells transformed by the plasmids; antibodies against the above polypeptides; and medicinal compositions containing the peptides or the antibodies.

### CROSS-REFERENCE TO RELATED APPLICATIONS

This is a continuation of U.S. patent application Ser. No. 11/593,037 filed Nov. 6, 2006, which is a Continuation application of U.S. patent application Ser. No. 10/404,438 filed Apr. 2, 2003 (abandoned), which is a Continuation application of U.S. patent application Ser. No. 09/582,419 filed Jun. 26, 2000 (abandoned), which is a National Stage Entry of PCT Application No. PCT/JP98/05952 filed Dec. 25, 1998. The entire disclosures of the prior applications, are considered part of the disclosure of the accompanying continuation application and are hereby incorporated by reference.

### TECHNICAL FIELD

The present invention relates to novel polypeptides, a process for preparation thereof, cDNAs encoding the polypeptide, vectors containing the cDNA, host cells transformed with the vector, antibodies against the polypeptide, and pharmaceutical compositions containing the polypeptide or the antibody.

### TECHNICAL BACKGROUND

Until now, when one skilled in the art intends to obtain a particular polypeptide or a cDNA encoding it, he/she generally utilizes methods by confirming an aimed biological activity in a tissue or in a cell medium, isolating and purifying the polypeptide and then cloning a gene or methods by “expression-cloning” with the guidance of the biological activity. However, physiologically active polypeptides in living body have often many kinds of activities. Therefore, it happens increasingly that after cloning a gene, the isolated gene is found to be identical to that encoding a polypeptide already known. In addition, some factors could be generated in only a very slight amount and/or under specific conditions and it makes difficult to isolate and to purify the factor and to confirm its biological activity.

Recent rapid developments in techniques for constructing cDNAs and sequencing techniques have made it possible to quickly sequence a large amount of cDNAs. By utilizing these techniques, a process, which comprises constructing cDNA library using various cells or tissues, cloning the cDNA at random, identifying the nucleotide sequences thereof, and expressing novel polypeptides encoded thereby, is now in progress. Although this process is advantageous in that a gene can be cloned and information regarding its nucleotide sequence can be obtained without any biochemical or genetic analysis, the target gene can be discovered thereby only accidentally in many cases.

The present inventors have studied a cloning method to isolate genes encoding proliferation and/or differentiation factors functioning in hematopoietic systems and immune systems. Focusing their attention on the fact that most of the secretory proteins, such as proliferation and/or differentiation factors (for example, various cytokines etc.) and membrane proteins such as receptors thereof (hereafter these proteins will be referred to generally as secretory proteins and the like), have sequences called signal peptides in the N-termini, the inventors have conducted extensive studies on a process for efficiently and selectively cloning a gene encoding a signal peptide. Finally, we have successfully developed a method which can easily select a cDNA encoding a signal peptide (signal sequence trap (SST)) by using animal cells (See Japanese Published Patent Application No. Hei 6-315380). We also developed yeast SST method on the same conception. By the method based on the same conception using yeast (yeast SST method), genes encoding a signal peptide can be identified more easily and efficiently (See U.S. Pat. No. 5,536,637).

### DISCLOSURE OF THE PRESENT INVENTION

The present inventors have diligently performed certain investigation in order to isolate novel factors (polypeptides) useful for treatment, diagnosis and/or study, particularly, secretory proteins containing a secretory signal and membrane proteins.

From the result, using the above methods, the present inventors achieved to find novel secretory proteins and membrane proteins produced from cell lines and tissue, for example, human adult brain tissue, cell lines derived from human brain tissue, cell lines derived from human bone marrow, and human fetal liver, and cDNAs encoding them, and then completed the present invention.

The present invention provides cDNA sequences identified as clones OM007 and OMB096 which were isolated by the above yeast SST method using cDNA libraries prepared from human adult brain tissue. Clones OM007 and OMB096 were full-length cDNAs including full cDNA sequences encoding secretory proteins (represented as OM007 and OMB096 proteins, respectively).

It was indicated from the results of homology search for the public database of the nucleic acid sequences by using BLASTN and FASTA, and for the public database of the amino acid sequences by using BLASTX, BLASTP and FASTA, that there was no sequence identical to the polypeptide sequences of OM007 and OMB096 of the present invention and the nucleotide sequences encoding them. From these results, it was proved that the polypeptides of the present invention were new secretary proteins.

The present invention provides cDNA sequences identified as clones OAF0038-Leu and OAF038-Pro which were isolated by the above yeast SST method using cDNA libraries prepared from human adult born marrow (HAS303). Clones OAF0038-Leu and OAF038-Pro were full-length cDNAs including full cDNA sequences encoding membrane proteins (represented as OAF0038-Leu and OAF038-Pro proteins, respectively).

It was indicated from the results of homology search for the public database of the nucleic acid sequences by using BLASTN and FASTA, and for the public database of the amino acid sequences by using BLASTX, BLASTP and FASTA, that there was no sequence identical to the polypeptide sequences of OAF0038-Leu and OFA038-Pro of the present invention and the nucleotide sequences encoding them. From these results, it was proved that the polypeptides of the present invention were new membrane proteins.

The present invention provides a cDNA sequence identified as clone OR087H which was isolated by the above yeast SST method using cDNA libraries prepared from human fetal liver. Clone OR087H was a full-length cDNA including a full cDNA sequence encoding a secretory protein (represented as OR087H protein).

It was indicated from the results of homology search for the public database of the nucleic acid sequences by using BLASTN and FASTA, and for the public database of the amino acid sequences by using BLASTX, BLASTP and FASTA, that there was no-sequence identical to the polypeptide sequence of OR087H of the present invention and the nucleotide sequences encoding it. From these results, it was proved that the polypeptide of the present invention was a new secretary protein.

The present invention provides cDNA sequences identified as clones OA004-FG and OA004-LD which were isolated by the above yeast SST method using cDNA libraries prepared from a human glioblastoma cell line T98G. Clones OA004-FG and OA004-LD were full-length cDNAs including full cDNA sequences encoding membrane proteins (represented as OA004-FG and OA004-LD proteins, respectively).

It was indicated from the results of homology search for the public database of the nucleic acid sequences by using BLASTN and FASTA, and for the public database of the amino acid sequences by using BLASTX, BLASTP and FASTA, that there was no sequence identical to the polypeptide sequences of OA004-FG and OA004-LD of the present invention and the nucleotide sequences encoding them. From these results, it was proved that polypeptides of the present invention were new membrane proteins.

That is to say, the present invention relates to:

(1) a polypeptide comprising the amino acid sequence of SEQ ID NOs: 1, 4, 7, 10, 13, 16 or 19,
(2) a cDNA encoding the polypeptide described in (1),
(3) a cDNA comprising the nucleotide sequence of SEQ ID NOs: 2, 5, 8, 11, 14, 17 or 20, and
(4) a cDNA comprising the nucleotide sequence of SEQ ID NOs: 3, 6, 9, 12, 15, 18 or 21.

### DETAILED DESCRIPTION OF THE PRESENT INVENTION

The present invention relates to a polypeptide in substantially purified form comprising the amino acid sequence shown in SEQ ID NOs: 1, 4, 7, 10, 13, 16 or 19, a homologue thereof, a fragment thereof, or a homologue of the fragment.

Further, the present invention relates to a cDNA encoding the above polypeptide. More particularly, the invention relates to a cDNA comprising the nucleotide sequence shown in SEQ ID NOs: 2, 5, 8, 11, 14, 17 or 20, and a cDNA containing a fragment which is selectively hybridized to a cDNA comprising the nucleotide sequence shown in SEQ ID NOs: 2, 3, 5, 6, 8, 9, 11, 12, 14, 15, 17, 18, 20 or 21. The cDNA capable of hybridizing to the cDNA includes the contemporary sequence of the above sequence. The conditions of the hybridizing are preferably stringent.

The polypeptide in substantially purified form comprising the amino acid sequence shown in SEQ ID NOs: 1, 4, 7, 10, 13, 16 or 19 will generally comprise the polypeptide in a preparation in which 90% or more, e.g., 95%, 98% or 99%, of the polypeptide in the preparation is that of the SEQ ID NOs: 1, 4, 7, 10, 13, 16 or 19.

The homologue of the polypeptide comprising the amino acid sequence shown in SEQ ID NOs: 1, 4, 7, 10, 13, 16 or 19 will be generally at least 70%, preferably at least 80 or 90%, and more preferably at least 95%, homologous to the polypeptide over a region of at least 20, preferably at least 30, for instance 40, 60 or 100 or more, contiguous amino acids. Such a polypeptide homologue will be referred to as “a polypeptide of the present invention”.

Also, the fragment of the polypeptide comprising the amino acid sequence shown in SEQ ID NOs: 1, 4, 7, 10, 13, 16 or 19 or its homologues will be generally at least 10, preferably at least 15, for example 20, 25, 30, 40, 50 or 60, amino acids in length.

The cDNA capable of selectively hybridizing to the cDNA comprising the nucleotide sequence shown in SEQ ID NOs: 2, 3, 5, 6, 8, 9, 11, 12, 14, 15, 17, 18, 20 or 21 will be generally at least 70%, preferably at least 80 or 90%, and more preferably at least 95%, homologous to the cDNA over a region of at least 20, preferably at least 30, for instance 40, 60 or 100 or more, contiguous nucleotides. Such a cDNA will be referred to as “a cDNA of the present invention”.

The fragment of the cDNA comprising the nucleotide sequence shown in SEQ ID NO. 2, 3, 5, 6, 8, 9, 11, 12, 14, 15, 17, 18, 20 or 21 will be at least 10, preferably at least 15, for example 20, 25, 30 or 40, nucleotides in length. Such a fragment will be also referred to as “a cDNA of the present invention”.

A further embodiment of the present invention provides replication and expression vectors carrying the cDNA of the present invention. The vectors may be, for example, plasmid, virus or phage vectors provided with an origin of replication, optionally a promoter for the expression of the cDNA and optionally a regulator of the promoter. The vectors may contain one or more selectable marker genes, for example an ampicillin resistance gene. The vectors may be used in vitro, for example, in the production of RNA corresponding to the cDNA, or the transformation of a host cell.

A further embodiment of the present invention provides host cells transformed with the vectors for the replication and expression of the cDNA of the present invention, including the cDNA comprising the nucleotide sequence shown in SEQ ID NOs: 2, 3, 5, 6, 8, 9, 11, 12, 14, 15, 17, 18, 20 or 21 or an open reading frame thereof. The cells include, for example, bacteria, yeast, insect cells, and mammalian cells.

A further embodiment of the present invention provides a method of producing a polypeptide which comprises culturing host cells of the present invention under conditions effective to express a polypeptide of the present invention. Preferably, in addition, such a method is carried out under conditions in which the polypeptide of the present invention is expressed and then produced from the host cells.

The cDNA of the present invention may also be inserted into the vectors described above in an antisense orientation in order to prove for the production of an antisense RNA. Such an antisense RNA may be used in a method of controlling the levels of the polypeptide of the present invention in a cell.

The present invention also provides monoclonal or polyclonal antibodies against the polypeptides of the present invention. The invention further provides a process for the production of monoclonal or polyclonal antibodies to the polypeptides of the present invention. The monoclonal antibodies may be prepared by common hybridoma technology using the polypeptides of the present invention or fragments thereof, as an immunogen. The polyclonal antibodies may also be prepared by common means which comprise inoculating host animals (for example, a rat, a rabbit, etc.) with the polypeptides of the present invention and recovering immune serum.

The present invention also provides pharmaceutical compositions comprising the polypeptide of the present invention or an antibody thereof, and a pharmaceutically acceptable diluent and/or carrier.

The polypeptides of the present invention specified in (1) include polypeptides in which a part of the amino acid sequence is lacking (e.g., a polypeptide comprised of only the essential sequence for revealing a biological activity from the amino acid sequence shown in SEQ ID NO: 1), polypeptides in which a part of their amino acid sequence is replaced by other amino acids (e.g., those replaced by an amino acid having a similar property), and polypeptides in which other amino acids are added or inserted into a part of their amino acid sequence, as well as those comprising the amino acid sequence shown in SEQ ID NOs: 1, 4, 7, 10, 13, 16 or 19.

As is well-known, there are one to six codons that encodes one amino acid (for example, one kind of codon for Met, and six codons for Leu). Accordingly, the nucleotide sequence of cDNA can be changed in order to encode a polypeptide having the same amino acid sequence.

The cDNAs of the present invention specified in (2) include a group of every nucleotide sequence encoding the polypeptide (1) shown in SEQ ID NOs: 1, 4, 7, 10, 13, 16 or 19. There is a probability that the yield of a polypeptide is improved by changing a nucleotide sequence.

The cDNAs specified by SEQ ID NOs: 2, 5, 8, 11, 14, 17 or 20 in (3) are the embodiment of the cDNAs shown in (2), and indicate the natural form of the sequence.

The cDNAs shown by SEQ ID NOs: 3, 6, 9, 12, 15, 18 or 21 in (4) indicates the sequence of the cDNAs specified in (3) with a natural non-translated region.

The cDNA having the nucleotide sequence shown in SEQ ID NOs: 3, 6, 9, 12, 15, 18, or 21 is prepared by the following method:

Brief description of Yeast SST method (see U.S. Pat. No. 5,536,637) is as follows.

Yeast, such as Saccharomyces cerevisiae, should secrete invertase into the medium in order to take sucrose or raffinose as a source of energy or carbon (the invertase is an enzyme to cleave raffinose into sucrose and melibiose, and sucrose into fructose and glucose). It is known that many known mammalian signal sequences make yeast secrete its invertase. From these knowledge, SST method was developed as a screening method to find a novel signal peptide which makes it possible to secrete yeast invertase from mammalian cDNA library using yeast growth on a raffinose medium as a marker.

Non-secretory type invertase gene SUC2 (GENBANK, Accession No. V01311) that lacks initiation codon ATG was inserted to a yeast expression vector to prepare vector pSUC2 for yeast SST. Into this expression vector, an expression promoter (ADH promoter) and a terminator (ADH terminator) both derived from AAH5 plasmid (Gammerer, Methods in Enzymol., 101, 192-201, 1983)) were inserted, and 2μ ori, TRP1, ColE1 ori and an ampicillin resistance gene were inserted as a yeast replication origin, a yeast selective marker, an E. coli replication origin and a drug resistance marker, respectively.

A mammalian cDNA was inserted into the upstream of SUC2 gene to prepare yeast SST cDNA library. This library was transformed into yeast that lacks secretory type invertase. If the inserted mammalian cDNA encodes a signal peptide, the yeast could survive in raffinose medium as a result of restoring secretion of invertase. Thus, it is possible to identify a novel signal peptide rapidly and easily by culturing expressed yeast colonies, preparing plasmids and determining the nucleotide sequence of the insert cDNAs.

Preparation of yeast SST cDNA library is as follows:

(1) mRNA is isolated from the targeted cells, double-strand synthesis is performed by using a random primer with a certain restriction enzyme (enzyme I) recognition site to obtain a double-strand cDNA,
(2) the obtained double-strand cDNA is ligated to an adapter containing a certain restriction endonuclease (enzyme II) recognition site, which is different from enzyme I, digested with enzyme I and fractionated to an appropriate size,
(3) the obtained cDNA fragment is inserted into a yeast expression vector on the upstream region of an invertase gene in which a signal peptide is deleted for transformation.

Detailed description of each step is as follows:

(1) mRNA is isolated from mammalian organs and cell lines stimulate them with an appropriate stimulator, if necessary) by known methods (Molecular Cloning (Sambrook, J., Fritsch, E. F. and Maniatis, T., Cold Spring Harbor Laboratory Press, 1989) or Current Protocol in Molecular Biology (F. M. Ausubel et al, John Wiley & Sons, Inc.) unless otherwise described hereinafter).

HAS303 (human bone marrow stroma cell line: provided from Professor Keisuke Sotoyama, Dr. Makoto Aizawa of First Medicine, Tokyo Medical College; see J. Cell. Physiol., 148, 245-251, 1991 and Experimental Hematol., 22, 482-487, 1994), human glioblastoma cell line TG98G (ATCC No. CRL-1690), and human fetal liver (CLONTECH, #CL6527-1) are chosen as a cell line. Human adult brain is chosen as a tissue source. Double-strand cDNA synthesis using random primer is performed by known methods.

Any sites may be used as restriction endonuclease recognition site I which is linked to an adapter and restriction endonuclease recognition site II which is used in step (2) if both sites are different from each other. Preferably, XhoI is used as enzyme I and EcoRI as enzyme II.

In step (2), the cDNA is blunt-ended with T4 DNA polymerase, ligated to enzyme II adapter, digested with enzyme I, and fractionized to cDNAs of 300 to 800 by with agarose-gel electrophoresis (AGE). As mentioned above, any enzyme may be used as enzyme II if it is different from enzyme I.

In step (3), cDNA fragments obtained in step (2) are inserted into a yeast expression plasmid vector on the upstream region of an invertase gene in which a signal peptide is deleted. E. coli was transformed with the expression vector. Many vectors are known as the yeast expression plasmid vector. For example, YEp24 which is also functioned in E. coli is used. Preferably, pSUC2 as described above is used.

Many host E. coli strains are known for transformation, preferably DH10B competent cell is used. Any known transformation method is available, preferably it is performed by electropolation method. The transformant is cultured by conventional methods to obtain cDNA library for yeast SST method.

However, the cDNA fragment is not inserted to all clones in this cDNA library. Further, all of the gene fragments do not encode unknown (novel) signal peptides. It is therefore necessary to screen a gene fragment encoding an unknown signal peptide from the library. Therefore, screening of fragments containing a sequence encoding a signal peptide is performed by transformation of the cDNA library into Saccharomyces cerevisiae having no invertase gene (e.g., YT455 strain) or a strain which artificially lacks an invertase gene (it may be prepared by known methods.).

Transformation of yeast is performed by known methods, e.g., lithium acetate method. A transformant is cultured in a selective medium, then transferred to a medium containing raffinose as a carbon source. Survival colonies are selected and then a plasmid is recovered. Survival colonies using raffinose as a carbon source indicates that some signal peptide of a secretory protein was inserted to this clone.

As for isolated positive clones, the nucleotide sequence is determined. As to a cDNA encoding an unknown protein, a full-length clone may be isolated by using the cDNA fragment as a probe and then the full-length nucleotide sequence was determined. These manipulations are performed by known methods.

Once the nucleotide sequences shown in SEQ ID NOs: 2, 5, 8, 11, 14, 17 or 20 are determined partially or preferably fully, it is possible to obtain cDNAs encoding the proteins of the present invention present in mammals or cDNAs encoding a homologue or subset of the proteins of the present invention. cDNA library or mRNA derived from mammals was screened by PCR with a synthesized oligonucleotide having an appropriate nucleotide sequence or by hybridization with a fragment having an appropriate nucleotide sequence as a probe. It is possible to obtain cDNAs encoding other mammalian homologue protein from other mammalian cDNA library or its genome library.

If the cDNA obtained above contains a nucleotide sequence of a cDNA fragment obtained by SST (or a consensus sequence thereof), it will be thought that the cDNA encodes the signal peptide. So it is clear that the cDNA will be full-length or almost full (all signal peptides exist at N-termini of a protein and are encoded at 5′-temini of the open reading frame of the cDNA).

The full-length may be confirmed by Northern analysis with the cDNA as a probe according to known methods. It is thought that the cDNA has an almost complete length, if the length of the cDNA is almost the same as the length of the mRNA obtained in the hybridizing band.

The proteins of the present invention include both a full-length type and a mature type. The full-length type and the mature type of the proteins are shown by SEQ ID NOs: 1, 4, 7, 10, 13, 16 and 19. These mature proteins can be obtained by expressing full-length cDNAs shown by SEQ ID NOs: 3, 6, 9, 12, 15, 18 and 21 in appropriate mammalian cells or other host cells. The sequence of the matured protein can be predicted from the amino acid sequence of the full-length type.

Once the nucleotide sequence shown in SEQ ID NOs: 2, 5, 8, 11, 14, 17 or 20 is determined, the cDNAs of the present invention can be obtained by chemical synthesis, or by hybridization making use of nucleotide fragments which have been chemically synthesized as a probe. Furthermore, the cDNAs of the present invention can be obtained in a desired amount by transforming a vector cDNA that contains the cDNA into a proper host, and culturing the transformant.

The polypeptides of the present invention may be prepared by, for example:

(1) isolating and purifying such polypeptides from an organism or a cultured cell,
(2) chemical peptide synthesis, or
(3) using recombinant cDNA technology, preferably, by the method described in (3) in an industrial production.

Examples of expression system (host-vector system) for producing a polypeptide by using recombinant cDNA technology are the expression systems of bacteria, yeast, insect cells and mammalian cells.

In the expression, for example, in E. coli, the expression vector is prepared by adding the initiation codon (ATG) to 5′-end of a cDNA encoding a mature protein, connecting the cDNA thus obtained to the downstream of a proper promoter (e.g., trp promoter, lac promoter, λPL promoter, T7 promoter etc.), and then inserting it into a vector (e.g., pBR322, pUC18, pUC19 etc.) which functions in an E. coli strain.

Then, an E. coli strain (e.g., E. coli DH1 strain, E. coli JM109 strain, E. coli HB101 strain, etc.) which is transformed with the expression vector described above may be cultured in an appropriate medium to obtain the desired polypeptide. When a signal sequence of bacteria (e.g., signal sequence of pel B) is utilized, the desired polypeptide may also be released in periplasm. Furthermore, a fusion protein with other polypeptide may also be produced.

In the expression of the polypeptides in mammalian cells, for example, the expression vector is prepared by inserting the cDNA encoding the nucleotide shown in SEQ ID NOs: 2, 5, 8, 11, 14, 17 or 20 into the downstream of a proper promoter (e.g., SV40 promoter, LTR promoter, metallothionein promoter, etc.) in a proper vector (e.g., retrovirus vector, papilloma virus vector, vaccinia virus vector, SV40 vector, etc.). A proper mammalian cell (e.g., monkey COS-7 cell, Chinese hamster CHO cell, mouse L cell, etc.) is transformed with the expression vector thus obtained, and then the transformant is cultured in a proper medium to express the protein (polypeptide) of the present invention by the following method depending on whether it is a secretory protein or a membrane protein.

In case of a secretory protein as the protein of the present invention, the aimed polypeptide was expressed in the supernatant of the cells. In addition, a fusion protein may be prepared by conjugating a cDNA fragment encoding other polypeptide, for example, the Fc portion of an antibody.

On the other hand, in case of a membrane protein as the protein of the present invention, the aimed polypeptide was expressed on the cell membrane. A cDNA encoding the nucleotide sequence of SEQ ID NO. 9, 12, 18 or 21 with deletion of the extracellular region was inserted into the above vector, transfected into the adequate mammalian cells to secret the aimed soluble polypeptide in the culture medium. In addition, a fusion protein may be prepared by conjugating a cDNA fragment encoding the mutant with deletion of the extracellular region and other polypeptide, for example, the Fc portion of an antibody.

The polypeptide available by the way described above can be isolated and purified by conventional biochemical method.

### INDUSTRIAL APPLICABILITY

It is considered that the polypeptides of the present invention and cDNAs encoding them will show one or more effects or biological activities (including those which relates to assays cited below). The effects or biological activities described in relation to the proteins of the present invention are provided by administration or use of the protein or by administration or use of a cDNA molecule which encodes the protein (e.g., vector suitable for gene therapy or cDNA introduction).

### Cytokine Activity and Cell Proliferation/Differentiation Activity:

The protein of the present invention may exhibit cytokine activity, cell proliferation (either inducing or inhibiting) or cell differentiation (either inducing or inhibiting) activity, or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of the polypeptide of the present invention is evidenced by any one of a number of routine factor dependent cell proliferation assays for cell lines.

### Immune Stimulating/Suppressing Activity:

The protein of the present invention may also exhibit immune stimulating or immune suppressing activity. The protein of the present invention may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral infection, such as HIV, as well as bacterial or fungal infections, or may result from autoimmune disorders. More specifically, infectious diseases causes by viral, bacterial, fungal or other infection may be treatable using the protein of the present invention, including infections by HIV, hepatitis viruses, herpes viruses, mycobacteria, leshmania, malaria, and various fungal infections, such as candida. Of course, in this regard, the protein of the present invention may also be useful where a boost to the immune system generally would be indicated, i.e., in the treatment of a cancer.

The protein of the present invention may be useful in the treatment of allergic reactions and conditions, such as asthma or other respiratory problems. The protein of the present invention may also be useful in the treatment of the other conditions required to suppress the immuno system (for example, asthma or respiratory disease).

The protein of the present invention may also suppress chronic or acute inflammation, for example, that associated with infection, such as septic shock or systemic inflammatory response syndrome (SIRS), inflammatory bowel disease, Crohn's disease, or resulting from over production of cytokines, such as TNF or IL-1, wherein the effect was demonstrated by IL-11.

### Hematopoiesis Regulating Activity:

The protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis. The biological activities are concerned with the following all or some example(s), e.g., in supporting the growth and proliferation of erythroid progenitor cells alone or in combination with other cytokines, thereby indicating utility, for example, in treating various anemia or for use in conjunction with irradiation/chemotherapy to stimulate the production of erythroid precursors and/or erythroid cells; in supporting the growth and proliferation of myeloid cells, such as granulocytes and monocytes/macrophages, (i.e., traditional CSF activity) useful, for example, in conjunction with chemotherapy to prevent or treat consequent myelosuppression; in supporting the growth and proliferation of megakaryocytes and consequently of platelets thereby allowing prevention or treatment of various platelet disorders, such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stern cell disorders (such as those usually treated with transplantation, including, without limitation, aplastic anemia and paroxysmal nocturnal hemoglobinuria), as well as in repopulating the stem cell compartment post irradiation/chemotherapy, either in vitro or ex vivo (i.e., in conjunction with bone marrow transplantation) as normal cells or genetically manipulated for gene therapy.

The activity of the protein of the present invention may, among other means, be measured by the following methods:

### Tissue Generation/Regeneration Activity:

The protein of the present invention may also have utility in compositions used for bone, cartilage, tendon, ligament and/or nerve tissue growth or regeneration, as well as for wound healing and tissue repair, and in the treatment of burns, incisions and ulcers.

The protein of the present invention, which induces cartilage and/or bone growth in circumstances where bone is not normally formed, may be applied to the healing of bone fractures and cartilage damage or defects in humans and other animals. Such a preparation employing the protein of the present invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

The protein of the present invention may also be used in the treatment of periodontal disease, and in other tooth repair processes. Such agents may provide an environment to attract bone-forming cells, stimulate growth of bone-forming cells or induce differentiation of progenitors of bone-forming cells. The protein of the present invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. The protein of the present invention, which induces tendon/ligament-like tissue or other tissue formation in circumstances where such tissue is not normally formed, may be applied to the healing of tendon or ligament tears, deformities and other tendon or ligament defects in humans and other animals. Such a preparation employing the protein inducing a tendon/ligament-like tissue may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon Ligament cells or progenitors ex vivo for return in vitro (in vivo) to effect tissue repair. The compositions of the present invention may also be useful in the treatment of tendinitis, Carpal tunnel syndrome and other tendon or ligament defects. The compositions may also include an appropriate matrix and a sequestering agent which is well known in the art as well as a carrier.

The protein of the present invention may also be useful for proliferation of neural cells and for regeneration of nerve and brain tissue. i.e. for the treatment of central and peripheral nervous system diseases and neuropathies. as well as mechanical and traumatic disorders, which involve degeneration, death or trauma to neural cells or nerve tissue. More specifically, the protein of the present invention may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the invention include mechanical and traumatic disorders, such as spinal cord disorders, head trauma and cerebrovascular diseases such as stroke. Peripheral neuropathies resulting from chemotherapy or other medical therapies may also be treatable using the protein of the present invention.

It is expected that the protein of the present invention may also exhibit activity for generation of other tissues, such as organs (including, for example, pancreas, liver, intestine, kidney, skin, endothelium), muscle (smooth, skeletal or cardiac) and vascular (including vascular endothelium) tissue, or for promoting the proliferation of cells comprising such tissues. A part of the desired effects may be by inhibition of fibrotic scarring to allow normal tissue to regenerate.

The protein of the present invention may also be useful for gut protection or regeneration and treatment of lung or liver fibrosis, reperfusion injury in various tissues, and conditions resulting from systemic cytokine damage.

### Activin/Inhibin Activity:

The protein of the present invention may also exhibit activin- or inhibin-related activities. Inhibins are characterized by their ability to inhibit the release of follicle stimulating hormone (FSH), while activins are characterized by their ability to stimulate the release of follicle stimulating hormone (FSH). Thus, the protein of the present invention alone or in heterodimers with a member of the inhibin a family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the present invention, as a homodimer or as a heterodimer with other protein subunits of the inhibin-β group, may be useful as a fertility inducing therapeutic, based upon the ability of activin molecules in stimulating FSH release from cells of the anterior pituitary (See U.S. Pat. No. 4,798,885). The protein of the present invention may also be useful for advancement of the onset of fertility in sexually immature mammals, so as to increase the lifetime reproductive performance of domestic animals such as cows, sheep and pigs.

### Chemotactic/Chemokinetic Activity:

The protein of the present invention may have chemotactic or chemokinetic activity, e.g., functioning as a chemokine, for mammalian cells, including, for example, monocytes, neutrophils, T-cells, mast cells, eosinophils and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting site.

If a protein or peptide can stimulate, directly or indirectly, the directed orientation or movement of such cell population, it has chemotactic activity for a particular cell population. Preferably, the protein or peptide has the ability to directly stimulate directed movement of cells. Whether a particular protein has chemotactic activity for a population of cells can be readily determined by employing such protein or peptide in any known assay for cell chemotaxis.

### Hemostatic and Thrombolytic Activity:

The protein of the present invention may also exhibit hemostatic or thrombolyic activity. As a result, such a protein is expected to be useful in treatment of various coagulation disorders (including hereditary disorders, such as hemophilias) or to enhance coagulation and other hemostatic events in treating wounds resulting from trauma, surgery or other causes. The protein of the present invention may also be useful for dissolving or inhibiting formation of thromboses and for treatment and prevention of conditions resulting from, for example, infarction, stroke, etc.

### Receptor/Ligand Activity:

The protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including cellular adhesion molecules such as Selectins, Integrins and their ligands) and receptor/ligand pairs involved in antigen presentation, antigen recognition and development of cellular and humoral immune responses. Receptors and ligands are also useful for screening of potential peptide or small molecule inhibitors of the relevant receptor/ligand interaction. The protein of the present invention (including, without limitation, fragments of receptors and ligands) per se may be useful as inhibitors of receptor/ligand interactions.

### Other Activity:

The protein (polypeptide) of the present invention may also exhibit one or more of the following additional activities or effects: inhibiting growth of or killing the infecting agents including bacteria, viruses, fungi and other parasites; suppressing or enhancing body characteristics including height, weight, hair color, eye color, skin, other tissue pigmentation, size of organs, for example, breast augmentation, diminution etc.; effecting elimination of dietary fat, protein, carbohydrate; effecting behavioral characteristics including appetite, libido, stress, cognition (including cognitive disorders), depression and violent behaviors; providing analgesic effects or other pain reducing effects; promoting differentiation and growth of embryonic stem cells in lineages other than hematopoietic lineages; in the case of enzymes, correcting deficiencies of the enzyme and treating deficiency-related diseases.

The protein with above activities, is suspected to have following functions by itself or interaction with its ligands or receptors or association with other molecules. For example, proliferation or cell death of B cells, T cells and/or mast cells; specific induction by promotion of class switch of immunoglobulin genes; differentiation of B cells to antibody-forming cells; proliferation, differentiation, or cell death of precursors of granulocytes; proliferation, differentiation, or cell death of precursors of monocytes-macrophages; proliferation, of up regulation or cell death of neutrophils, monocytes-macrophages, eosinophils and/or basophils; proliferation, or cell death of precursors of megakaryocytes; proliferation, differentiation, or cell death of precursors of neutrophils; proliferation, differentiation, or cell death of precursors of T cells and B cells; promotion of production of erythrocytes; sustainment of proliferation of erythrocytes, neutrophils, eosinophils, basophils, monocytes-macrophages, mast cells, precursors of megakaryocyte; promotion of migration of neutrophils, monocytes-macrophages, B cells and/or T cells; proliferation or cell death of thymocytes; suppression of differentiation of adipocytes; proliferation or cell death of natural killer cells; proliferation or cell death of hematopoietic stem cells; suppression of proliferation of stem cells and each hematopoietic precursor cells; promotion of differentiation from mesenchymal stem cells to osteoblasts or chondrocytes, proliferation or cell death of mesenchymal stem cells, osteoblasts or chondrocytes and promotion of bone absorption by activation of osteoclasts and promotion of differentiation from monocytes to osteoclasts.

The polypeptide of the present invention is also suspected to function to nervous system, so expected to have functions below; differentiation to kinds of neurotransmitter-responsive neurons, survival or cell death of these cells; promotion of proliferation or cell death of glial cells; spread of neural dendrites; survival or cell death of gangriocytes; proliferation, promotion of differentiation, or cell death of astrocytes; proliferation, survival or cell death of peripheral neurons; proliferation or cell death of Schwann cells; proliferation, survival or cell death of motoneurons.

Furthermore, in the process of development of early embryonic, the polypeptide of the present invention is expected to promote or inhibit the organogenesis of epidermis, brain, backbone, and nervous system by induction of ectoderm, that of notochord connective tissues (bone, muscle, tendon), hemocytes, heart, kidney, and genital organs by induction of mesoderm, and that of digestive apparatus (stomach, intestine, liver, pancreas), respiratory apparatus (lung, trachea) by induction of endoderm. In adult, also, this polypeptide is thought to proliferate or inhibit the above organs.

Therefore, the polypeptide of the present invention itself is expected to be used as an agent for the prevention or treatment of disease of progression or suppression of immune, nervous, or bone metabolic function, hypoplasia or overgrowth of hematopoietic cells: for example, inflammatory disease (rheumatism, ulcerative colitis, etc.), decrease of hematopoietic stem cells after bone marrow transplantation, decrease of leukocytes, platelets, B-cells, or T-cells after radiation exposure or chemotherapeutic dosage against cancer or leukemia, anemia, infectious disease, cancer, leukemia, AIDS, bone metabolic disease (osteoporosis etc.), various degenerative disease (Alzheimer's disease, multiple sclerosis, etc.), or nervous lesion.

In addition, since the polypeptide of the present invention is thought to induce the differentiation or growth of organs derived from ectoderm, mesoderm, and endoderm, this polypeptide is expected to be an agent for tissue repair (epidermis, bone, muscle, tendon, heart, kidney, stomach, intestine, liver, pancreas, lung, trachea, etc.).

By using polyclonal or monoclonal antibodies against the polypeptide of the present invention, quantitation of the polypeptide in the body can be performed. It can be used in the study of relationship between this polypeptide and disease or diagnosis of disease, and so on. Polyclonal and monoclonal antibodies can be prepared using the polypeptide or its fragment as an antigen by conventional methods.

Identification, purification or molecular cloning of known or unknown proteins which bind the polypeptide of the present invention (preferably polypeptide of extracellular domain) can be performed using the polypeptide of the present invention by, for example, preparation of the affinity-column.

Identification of the downstream signal transmission molecules which interact with the polypeptide of the present invention in cytoplasma and molecular cloning of the gene can be performed by west-western method using the polypeptide of the present invention (preferably polypeptide of transmembrane region or intracellular domain), or by yeast two-hybrid system using the cDNA (preferably cDNA encoding the transmembrane region or cytoplasmic domain of the polypeptide).

Agonists/antagonists of this receptor polypeptide and inhibitors between receptor and signal transduction molecules can be screened using the polypeptide of the present invention.

The cDNAs of the present invention are useful not only the important and essential template for the production of the polypeptide of the present invention which is expected to be largely useful, but also be useful for diagnosis or therapy (for example, treatment of gene lacking, treatment to stop the expression of the polypeptide by antisense cDNA (mRNA)). Genomic cDNA may be isolated with the cDNA of the present invention, as a probe. In the same manner, a human gene encoding a related polypeptide which can be highly homologous to the cDNA of the present invention or gene encoding a polypeptide highly homologous to the polypeptide of the present invention and a gene of animals excluding mouse encoding a polypeptide which can be highly homologous to the polypeptide of the present invention, also may be isolated.

### Application to Medicaments:

The polypeptide of the present invention or the antibody specific for the polypeptide of the present invention is administered systemically or topically and in general orally or parenterally, preferably parenterally, intravenously and intraventricularly, for preventing or treating the diseases.

The doses to be administered depend upon age, body weight, symptom, desired therapeutic effect, route of administration, and duration of the treatment etc. In human adults, one dose per person is generally between 100 μg and 100 mg, by oral administration, up to several times per day, and between 10 μg and 100 mg, by parental administration, up to several times per day.

As mentioned above, the doses to be used depend upon various conditions. Therefore, there are cases in which doses lower than or greater than the ranges specified above may be used.

The compounds of the present invention may be administered as solid compositions, liquid compositions or other compositions for oral administration, as injections, liniments or suppositories etc. for parental administration.

Solid compositions for oral administration include compressed tablets, pills, capsules, dispersible powders, and granules. Capsules include soft and hard capsules.

In such compositions, one or more of the active compound(s) may be admixed with at least one inert diluent (such as lactose, mannitol, glucose, hydroxypropyl cellulose, microcrystalline cellulose, starch, polyvinylpyrrolidone, magnesium metasilicate aluminate, etc.). The compositions may also comprise, as is normal practice, additional substances other than inert diluents: e.g., lubricating agents (such as magnesium stearate etc.), disintegrating agents (such as cellulose calcium glycolate, etc.), stabilizing agents (such as human serum albumin, lactose etc.), and assisting agents for dissolving (such as arginine, asparaginic acid etc.).

The tablets or pills may, if desired, be coated with a film of gastric or enteric materials (such as sugar, gelatin, hydroxypropyl cellulose, hydroxypropylmethyl cellulose phthalate, etc.), or be coated with at least two films. And then, coating may include containment within capsules of absorbable materials such as gelatin.

Liquid compositions for oral administration include pharmaceutically-acceptable emulsions, solutions, syrups and elixirs. In such compositions, one or more of the active compound(s) may be contained in inert diluent(s) commonly used (purified water, ethanol etc.). Besides inert diluents, such compositions may also comprise adjuvants (such as wetting agents, suspending agents, etc.), sweetening agents, flavoring agents, perfuming agents, and preserving agents.

Other compositions for oral administration include spray compositions which may be prepared by known methods and which comprise one or more of the active compound(s). Spray compositions may comprise additional substances other than inert diluents: e.g., stabilizing agents (sodium sulfite etc.), isotonic buffer (sodium chloride, sodium citrate, citric acid, etc.). For preparation of such spray compositions, for example, the method described in the U.S. Pat. No. 2,868,691 or 3,095,355 may be used.

Injections for parental administration include sterile aqueous or non-aqueous solutions, suspensions and emulsions. In such compositions, one or more active compound(s) is or are admixed with at least one inert aqueous diluent(s) (distilled water for injection, physiological salt solution, etc.) or inert non-aqueous diluents(s) (propylene glycol, polyethylene glycol, olive oil, ethanol, POLYSOLBATE 80 (Trademark) etc.).

Injections may comprise additional compound other than inert diluents: e.g., preserving agents, wetting agents, emulsifying agents, dispersing agents, stabilizing agent (such as human serum albumin, lactose, etc.), and assisting agents, such as assisting agents for dissolving (arginine, asparaginic acid, etc.).

### BEST MODE CARRYING OUT THE INVENTION

The invention is illustrated by the following examples, but the invention is not limited thereto.

### Example 1

### Clone OM007

### Preparation of Poly(A)⁺RNA:

A total RNA was prepared from human adult brain tissue by TRIzol reagent (Trademark, marketed by GIBCO BRL Co.). Poly(A)⁺RNA was purified from the total RNA by mRNA Purification Kit (Trade name, marketed by Pharmacia Co.).

Preparation of Yeast SST cDNA Library:

A double strand cDNA was synthesized by Super Script Plasmid System for cDNA Synthesis and Plasmid Cloning (Trade name, marketed by GIBCOBRL Co.) with above poly(A)⁺RNA as template and random 9mer as primer which was containing XhoI site:

(SEQ ID NO: 22)
5′-CGATTGAATTCTAGACCTGCCTCGAGNNNNNNNNN-3.

The cDNA was ligated with EcoRI adapter by DNA ligation kit ver. 2 (Trade name, marketed by Takara Shuzo Co.; this kit was used in all ligating steps hereinafter.) and digested by XhoI. The cDNAs were separated by agarose-gel electrophoresis. 300 to 800 by cDNAs were isolated and were ligated to EcoRI/NotI site of pSUC2 (see U.S. Pat. No. 5,536,637). E. coli DH10B strains were transformed by pSUC2 with electropolation to obtain yeast SST cDNA library.

### Screening by SST Method and Determination of Nucleotide Sequence of SST Positive Clone:

Plasmids of the cDNA library were prepared. Yeast YTK12 strains were transformed by the plasmids with lithium acetate method (Current Protocols In Molecular Biology, 13.7.1). The transformed yeast were plated on a triptphan-free medium (CMD-Trp medium) for selection. The plate was incubated for 48 hour at 30° C. Replica of the colony (transformant) which was obtained by Accutran Replica Plater (Trade name, marketed by Schleicher & Schuell) were placed onto YPR plate containing raffinose for a carbon source, and the plate was incubated for 14 days at 30° C. After 3 days, each colony appeared was streaked on YPR plate again. The plates were incubated for 48 hours at 30° C. Single colony was inoculated to YPD medium and was incubated for 48 hours at 30° C. Then plasmids were prepared. An insert cDNA was amplified by PCR with two kind primers which exist on the end side of the cloning site on pSUC2 (sense strand primers were biotinylated). Biotinylated single strand of cDNAs were purified with Dynabeads (Trade name, marketed by DYNAL Co.) and the nucleotide sequences were determined. Sequencing was performed by Dye Terminator Cycle Sequencing Ready Reaction with DNA Sequencing kit (Trade name, marketed by Applied Biosystems Inc.) and the sequence was determined by DNA sequencer 373 (Applied Biosystems Inc.) (all sequencing hereafter was carried out with this method.).

We tried to carry out cloning of full-length cDNA which was proved to be new one according to the homology search for the obtained nucleotide sequences and deduced amino acid sequences in data base. We also confirmed that each cDNA contains a signal peptide in view of function and structure, by comparison with a known signal peptide and the deduced amino acid sequence.

Cloning of a Full-Length cDNA and Determination of Nucleotide Sequence:

A full-length cDNA was cloned using Marathon cDNA Amplification Kit (Trade name, marketed by Clontech Co.) according to 3′ RACE (Rapid Amplification of cDNA End) method. A double strand cDNA was prepared from the origin of each clone, i.e., poly(A)⁺RNA in human adult brain tissue. 27mer primer OM007-F3:

| | |
|---|---|
| 5′-AACTGCAGATCTTGGGACTCATCAGCC-3′ | (SEQ ID NO: 23) |

containing the deduced initiation ATG codon region based on the information of the nucleotide sequence obtained by SST, was prepared. PCR was performed with the primer and an adapter primer attached in the kit. Due to insufficient amplification of cDNA by only one-time PCR, 28mer primer OM007-F2:

| | |
|---|---|
| 5′-AAGAGGACATTGTTTTCATCATGGATGC-3′ | (SEQ ID NO: 24) |

was prepared additionally at 3′-end of OM007-F1 primer and then nested PCR was performed. A cDNA which was amplified with clone OM007 specifically was separated with agarose-gel electrophoresis, ligated to pT7 Blue-2 T-Vector (Trade name, marketed by Novagen Co.) and transfected into E. coli DH5a to prepare a plasmid. First, nucleotide sequences of 5′-end were determined, and the existence of nucleotide sequence OM007 SST cDNA was confirmed. A nucleotide sequence of full-length OM007 SST cDNA was determined and then the cDNA sequence shown in SEQ ID NO: 3 was obtained. An open reading frame was determined and deduced an amino acid sequence, and sequences shown in SEQ ID NOs: 1 and 2 were obtained.

It was indicated from the results of homology search for the public database of the nucleic acid sequences by using BLASTN and FASTA, and for the public database of the amino acid sequences by using BLASTX, BLASTP and FASTA, that there was no sequence identical to the polypeptide sequence of OM007 of the present invention and the nucleotide sequences encoding them. From these results, it was proved that polypeptide of the present invention was a new secretary protein. However, the search using BLASTX, BLASTP and FASTA revealed a significant homology between clone OM007 (region of 21st to 765th amino acid in SEQ ID NO: 1) and a region of 9th to 753rd amino acids chicken collapsin-2 (collapsin-2, (Gallus gallus), Genbank Accession U28240). Based on the homology, clone OM007 and semaphorin family to which collapsin belongs are expected to share at least some activity.

### Example 2

### Clone OMB096

In Example relating to clone OMB096 of the present invention, the same procedure as in Example of OM007 was used, except for the following points.

Cloning of a Full-Length cDNA and Determination of Nucleotide Sequence:

A full-length cDNA was cloned using Marathon cDNA Amplification Kit (Trade name, marketed by Clontech Co.) according to 3′ RACE method in the same manner as in Example of OM007. A double strand cDNA was prepared from the origin of each clone, i.e., poly(A)⁺RNA in human adult brain tissue. 27mer primer OMB096-F1:

| | |
|---|---|
| 5′-ACAACATGCACCACCAGTGGCTTCTGC-3′ | (SEQ ID NO: 25) |

containing the deduced initiation ATG codon region based on the information of the nucleotide sequence obtained by SST, was prepared. PCR was performed with the primer and an adapter primer attached in the kit. A cDNA which was amplified with clone OMB096 specifically was cloned in the same manner as in Example of OM007, a full nucleotide sequence was determined and then a cDNA sequence shown in SEQ ID NO: 6 was obtained. An open reading frame was determined and deduced an amino acid sequence, and sequences shown in SEQ ID NOs: 4 and 5 were obtained.

It was indicated from the results of homology search for the public database of the nucleic acid sequences by using BLASTN and FASTA, and for the public database of the amino acid sequences by using BLASTX, BLASTP and FASTA, that there was no sequence identical to the polypeptide sequence OMB096 of the present invention and the nucleotide sequences encoding it. From these results, it was proved that polypeptide of the present invention was a new secretary protein.

### Example 3

### Clones OAF038-Leu and OAF038-Pro

In Example relating to clones OAF038-Leu and OAF038-Pro of the present invention, the same procedure as in Example of OM007 was used, except for the following points.

### Preparation of Poly(A)⁺RNA:

A total RNA was prepared from human bone marrow stroma cell line HAS303 (provided from Prof. Keisuke Sotoyama, Dr. Makoto Aizawa, First Medicine, Tokyo Medical College) by TRIzol reagent (Trademark, marketed by GIBCOBRL Co.). Poly(A)⁺RNA was purified from the total RNA by mRNA Purification Kit (Trade name, marketed by Pharmacia Co.).

Cloning of a Full-Length cDNA and Determination of Nucleotide Sequence:

A full-length cDNA was cloned using Marathon cDNA Amplification Kit (Trade name, marketed by Clontech Co.) according to 3′ RACE method in the same manner as in Example of OM007. Double strand cDNA was prepared from the origin of each clone, i.e., poly(A)⁺RNA in HAS303. 28mer primer OAF038-F1:

| | |
|---|---|
| 5′-AGAATGTGGAGCCATTTGAACAGGCTCC-3′ | (SEQ ID NO. 26) |

containing the deduced initiation ATG codon region based on the information of the nucleotide sequence obtained by SST, was prepared. PCR was performed with the primer and an adapter primer attached in the kit. A cDNA which was amplified with clone OAF038 specifically was separated with recloning by the same method as in Example of OM007, a full-length nucleotide sequence was determined, and then cDNA sequences shown in SEQ ID NOs: 9 and 12 were obtained. Each clone was named OAF038-Leu and OAF308-Pro, respectively. Open reading frames were determined and deduced amino acid sequences, and sequences shown in SEQ ID NOs: 7 and 8 and NOs: 10 and 11, respectively, were obtained.

It was indicated from the results of homology search for the public database of the nucleic acid sequences by using BLASTN and FASTA, and for the public database of the amino acid sequences by using BLASTX, BLASTP and FASTA, that there was no sequence identical to the polypeptide sequences of OAF038-Leu and OAF038-Pro of the present invention and the nucleotide sequences encoding them. From these results, it was proved that polypeptides of the present invention were new membrane proteins. However, the search using BLASTX, BLASTP and FASTA revealed a significant homology between clones OAF038-Leu and OAF038-Pro (region of 5th to 343rd amino acids in SEQ ID NOs: 7 and 10) and rat MCA-32 protein (Rat MCA-32 protein, Genbank Accession U39546, a region of 42nd to 268th amino acids). Polypeptides OAF038-Leu and OAF038-Pro have Ig domain and SH2 domain at the extracell domain and the cytoplasmic domain, respectively, in the same manner as Rat MCA-32 protein. Based on the homology, clones OAF038-Leu and OAF038-Pro and the above Rat MCA-32 protein are expected to share at least some activity.

### Example 4

### Clone OR087H

In Example relating to clone OR087H of the present invention, the same procedure as in Example of OM007 was used, except for the following points.

### Preparation of Poly(A)⁺RNA:

We purchased poly(A)⁺RNA (CL6527-1) in human fetal liver from CLONTECH Co.

Cloning of a Full-Length cDNA and Determination of Nucleotide Sequence:

A full-length cDNA was cloned using Marathon cDNA Amplification Kit (Trade name, marketed by Clontech Co.) according to 3′ RACE method in the same manner as in Example of OM007. A double strand cDNA conjugating an adapter was prepared from the origin of each clone, i.e., poly(A)⁺RNA in human fetal liver according to the method of the kit. 27mer primer OR087H-F1:

| | |
|---|---|
| 5′-TGAAGCCCTTGTCCGTAAGCCTTGAAC-3′ | (SEQ ID NO: 27) |

containing the deduced initiation ATG codon region based on the information of nucleotide sequence obtained by SST, was prepared. PCR was performed with the primer and an adapter primer attached in the kit. A cDNA which was amplified with clone OR087H specifically was separated with recloning by the same method as in Example of OM007. A full-length nucleotide sequence was determined and then cDNA sequence shown in SEQ ID NO: 15 was obtained. An open reading frame was determined and deduced an amino acid sequence and sequences shown in SEQ ID NOs: 13 and 14 were obtained.

It was indicated from the results of homology search for the public database of the nucleic acid sequences by using BLASTN and FASTA, and for the public database of the amino acid sequences by using BLASTX, BLASTP and FASTA, that there was no sequence identical to the polypeptide sequence of OR087H of the present invention and the nucleotide sequences encoding them. From these results, it was proved that polypeptide of the present invention was a new secretary protein. However, the search using BLASTX, BLASTP and FASTA revealed a significant homology between clone OR087H (region of 1st to 115th amino acids in SEQ ID NO: 13) and human rapamycin- and FK506-binding protein (rapamycin- and FK506-binding protein (Homo sapiens), Genbank Accession M75099, a region of 1st to 116th amino acids). Based on the homology, clone OR087H and FK-binding protein family are expected to share at least some activity.

### Example 5

### Clones OA004-FG and OA004-LD

In Example relating to clones OA004-FG and OA004-LD of the present invention, the same procedure as in Example of OM007 was used, except for the following points.

### Preparation of Poly(A)⁺RNA:

Total RNA was prepared from human glioblastoma cell line T98G (ATCC No. CRL-1690) by TRIzol reagent (Trademark, marketed by GIBCOBRL Co.). Poly(A)⁺RNA was purified from the total RNA by mRNA Purification Kit (Trade name, marketed by Pharmacia Co.).

Cloning of a Full-Length cDNA and Determination of Nucleotide Sequence:

A full-length cDNA was cloned using GENETRAPPER cDNA Positive Selection System (Trade name, marketed by GIBCOBRL). First, dT-primed cDNA library was prepared from poly(A)⁺RNA in human glioblastoma cell line T98G using pSPORT1 plasmid (marketed by GIBCOBRL), as a vector, by Super Script Plasmid System for cDNA Synthesis and Plasmid Cloning (Trade name, marketed by GIBCOBRL). Next, after preparing 27 mer biotinylated primer OA004-F1:

(SEQ ID NO: 28)
5′ biotin-ATGCACATCTTCAAGCATGCTCAG-3′

based on the information of the nucleotide sequence obtained by SST, plasmids hybridized specifically with the biotinylated primer were recovered from the cDNA library according to the method of Gene Trapper Kit and then transfected into E coli DH10B. Colony hybridization with OA004 SST cDNA which was labeled with ³²P-dCTP, as a probe, was performed by using Random Primer DNA Labeling kit (Trade name, marketed by Takara Shuzo Co.) according to a known method to isolate a positive clone and to prepare a plasmid. After the nucleotide sequence at the 5′-side was determined to confirm that the nucleotide sequence of OA004 SST cDNA was present, a full-length nucleotide sequence was determined, cDNA sequences shown in SEQ ID NOs: 18 and 21 were obtained and named OA004-FG and OA004-LD, respectively. Furthermore, open reading frames were determined and deduced amino acid sequences, and sequences shown in SEQ ID NOs: 16 and 17 and NOs: 19 and 20, respectively, were obtained.

It was indicated from the results of homology search for the public database of the nucleic acid sequences by using BLASTN and FASTA, and for the public database of the amino acid sequences by using BLASTX, BLASTP and FASTA, that there was no sequence identical to the polypeptide sequences of OA004-FG and OA004-LD of the present invention and the nucleotide sequences encoding them. From these results, it was proved that polypeptides of the present invention were new secretary proteins. However, as a results of homology search, BLASTX, BLASTP and FASTA revealed a significant homology between clones OA004-FG and OA004-LD (region of 151st to 353rd amino acids in SEQ ID NOs: 16 and 19) and C. elegans 52.8 kD protein (Hypothetical 52.8 kD protein (Caenorhabdtis elegans), SwissProt Accession YJ95_CAEEL, region of 238th to 453rd amino acids). Moreover, they revealed a significant homology between clones OA004-FG and OA004-LD (region of 236th to 319th amino acids in SEQ ID NOs: 16 and 19) and human presenillin-2 (presenillin-2 (Homo sapiens), Genbank Accession A56993, region of 340th to 416th amino acids). Based on these homologies, clones OA004-FG and OA004-LD and presenillin family were expected to share at least some activity.

What is claimed is:

1. A polypeptide in substantially purified form comprising the amino acid sequence of SEQ ID NO:7 or 10.

2. A cDNA molecule encoding the polypeptide according to claim 1.

3. The cDNA molecule according to claim 2, comprising the nucleotide sequence of SEQ ID NO:8 or 11.

4. The cDNA molecule according to claim 2, comprising the nucleotide sequence of SEQ ID NO:9 or 12.

5. A replication or expression vector comprising the cDNA molecule according to claim 2.

6. A replication or expression vector comprising the cDNA molecule according to claim 3.

7. A replication or expression vector comprising the cDNA molecule according to claim 4.

8. A host cell transformed with the replication or expression vector according to claim 5.

9. A host cell transformed with the replication or expression vector according to claim 6.

10. A host cell transformed with the replication or expression vector according to claim 7.

11. A method for producing the polypeptide comprising the amino acid sequence of SEQ ID NO:7 or 10, said method comprising culturing a host cell according to claim 8 under conditions effective to express the polypeptide.

12. A method for producing the polypeptide comprising the amino acid sequence of SEQ ID NO:7 or 10, said method comprising culturing a host cell according to claim 9 under conditions effective to express the polypeptide.

13. A method for producing the polypeptide comprising the amino acid sequence of SEQ ID NO:7 or 10, said method comprising culturing a host cell according to claim 10 under conditions effective to express the polypeptide.
